(19) United States
(12) Patent Application Publication
(10) Pub. No.: US 2001/0056259 A1
(43) Pub. Date: Dec. 27, 2001

(54) Spring-powered infusion pump

(76) Inventors: David W. Skinkle, Denver, CO (US); Douglas J. McDowell, Erie, CO (US); Jerome M. Keane, Louisville, CO (US); L. Dean Stansbury, Wheat Ridge, CO (US); Andrew N. Lamborne, Denver, CO (US)

Correspondence Address: DORR CARSON SLOAN & BIRNEY, PC, 3010 EAST 6TH AVENUE, DENVER, CO, 80206

(21) Appl. No.: 09/898,769

(22) Filed: Jun. 29, 2001

Related U.S. Application Data
(60) Provisional application No. 60/114,206, filed on Dec. 29, 1998.

(30) Foreign Application Priority Data
Dec. 27, 1999 (US) PCT/US99/30890

(51) Int. Cl. A61M 1/00

(52) U.S. Cl. 604/151; 604/211; 604/118; 604/131; 604/135; 222/340

(57) ABSTRACT

A spring-powered infusion pump includes a syringe barrel 20 having two opposing openings 30, 55 forming two chambers 80, 90 between the first opening 30 and the plunger 40, and the second opening 55 and the plunger 40. The dispenser opening 30 has a one-way valve 35 to selectively release fluid retained within the first chamber 80. The second opening 55 is capped, and a spring 60 is compressed between the plunger 40 and syringe cap 50 within the second chamber 90. The spring 60 applies a force to the plunger 40 in the direction of the first opening 30. However, the one-way valve 35 retains the fluid within the first chamber 80, despite the force applied to the plunger 40, until a tubing set 70 equipped with an infuser connector 75 is attached to the dispenser opening 30 of the syringe barrel 20. The infuser connector 75 is insertable through the one-way valve 35 and thus provides a passageway for the fluid retained within the first chamber 80 of the syringe barrel 20. Once attached, the force from the spring 60 causes the plunger 40 to move toward the dispenser opening 30, thereby dispensing the fluid from the first chamber 80 through the infuser connector 75 and one-way valve 35 into the tubing set 75.

### RELATED APPLICATIONS

The present application is based in part on the Applicant's International Patent Application PCT/US99/30890, entitled “Spring-Powered Infusion Pump,” filed on Dec. 27, 1999, which is based on U.S. Provisional Patent Application Ser. No. 60/114,206, filed on Dec. 29, 1998.

### BACKGROUND OF THE INVENTION

1. Field of the Invention

The present invention relates generally to the field of spring-powered infusion pumps. More specifically, the present invention discloses a single-dose spring-powered infusion pump.

2. Statement of the Problem

Syringe-type infusers typically require the user to manually dispense the fluid contents (e.g., by pressing the syringe plunger). Thus, it is difficult to deliver a steady flow over time, especially when there is a large amount of fluid to be dispensed over a period of time. Spring-powered infusers, on the other hand, deliver fluid at a continuous rate, but require elaborate mechanisms, caps or clips to retain the fluid within the syringe because the plunger is under pressure.

Conventional spring-loaded infusers are initially loaded under pressure. Therefore, a secondary problem is created when the filling apparatus is disconnected from the infuser. If tubing is not immediately attached or the connection is not otherwise capped or clipped, the pressurized liquid will be lost, thus making the amount delivered inaccurate.

An additional problem associated with conventional spring-loaded infusers is limited delivery accuracy. The manufacturing process for springs introduces a significant amount of natural variation from spring to spring. This natural variation is a primary factor in limiting the accuracy with which a spring-loaded infuser can deliver liquids.

Syringe type infusers used in the past include the following:

| Inventor | Patent No. | Issue Date |
|---|---|---|
| Calhoun | 1,123,990 | Jan. 5, 1915 |
| Bessesen | 1,476,946 | Dec. 11, 1923 |
| Kollsman | 2,605,765 | Aug. 5, 1952 |
| Jinotti | 3,565,292 | Feb. 23, 1971 |
| Magoon, et al. | 4,312,347 | Jan. 26, 1982 |
| Genese | 4,381,006 | Apr. 26, 1983 |
| Vaillancourt | 4,813,937 | Mar. 21, 1989 |
| Chang | 4,991,742 | Feb. 12, 1991 |
| LeFevre | 4,997,420 | Mar. 5, 1991 |
| Reese | 5,078,679 | Jan. 7, 1992 |
| Vaillancourt | 5,100,389 | Mar. 31, 1992 |
| Zdeb | 5,135,500 | Aug. 4, 1992 |
| Ishikawa | 5,178,609 | Jan. 12, 1993 |
| Elson | 5,346,476 | Sep. 13, 1994 |
| Kriesel | 5,569,236 | Oct. 29, 1996 |
| McPhee | 5,599,315 | Feb. 4, 1997 |
| Ragsdale et al. | 5,607,395 | Mar. 4, 1997 |

Chang teaches an automatic drip bottle set. A cover and basin connect to hold a spring in the basin. The spring provides pressure urging the basin downward and applying uniform pressure on an expansion drip bottle.

LeFevre discloses a portable drug delivery device for delivering a drug in liquid form at a constant and self-regulated rate. A syringe having a spring-loaded piston in a cylinder forces the liquid out through a tubing having a restrictor in the length of tubing to impede flow and achieve a desired flow rate.

Zdeb teaches a self-driven pump device for delivering fluid at a relatively constant, controlled rate. A vacuum power means collapses under atmospheric pressure and drives a plunger to deliver fluid from the fluid storage means. The fluid storage means is filled by attaching a male luer opening to a female luer opening associated with the fluid storage means. As the male luer is pushed into the duck-bill valve, the tapered end portion opens to allow fluid to pass through the valve and into the fluid storage means. Fluid can then be delivered from the fluid storage means as the plunger moves under atmospheric pressure. A generally similar vacuum-powered infusion pump has been marketed by McKinley Medical, LLLP, of Wheat Ridge, Colo., as the “Outbound” disposable syringe infuser.

Calhoun discloses a type of syringe for dosing or inoculating animals. The syringe automatically discharges the contents when the user forces a small plunger inward, permitting a spring to draw a piston into the syringe barrel, thus forcing the contents out in a controlled manner.

Bessesen discloses a fluid-pressure device. When the aperture is closed and the barrel filled with fluid, pressure is created in the barrel by turning the handle to release the spring. When the barrel is emptied, the piston is retracted by turning the handle the opposite direction.

Kolisman teaches an automatic syringe. After removing a release cap from the end of the piston guide rod, a spring expands moving the piston toward a partition plug against the resistance of a viscous liquid in chamber 33. The liquid flows slowly through a capillary passage 36 into a chamber 32, which moves a piston 15 displacing fluid from the chamber 14 through an injection needle over a predetermined time.

Jinotti discloses an apparatus for holding a blood bag and causing the blood to be fed out of the bag. A piston is retracted by turning a handle to the desired position and then released so that the piston is under pressure created by the spring, which in turn forces blood out of the bag gradually and constantly.

Magoon et al. teach a positive-pressure drug releasing device. A chamber is filled with a liquid drug and placed under continuous positive pressure by a spring and plunger device. Fluid diffuses at a predetermined rate through a membrane opposite the plunger.

Genese discloses a continuous low flow rate fluid dispenser. Two spiral coiled springs move the driver member toward the abutment member, forcing the plunger stopper toward the nozzle portion expelling fluid from the syringe barrel at a slow and steady rate.

Vaillancourt ('937) teaches an ambulatory disposable infusion delivery system. Inflow of a fluid causes an elastomeric member attached to a piston to stretch, which pushes the fluid out of the bore when the tubing line is opened. The housing is provided with a discharge fluid conduit and a restrictor controlling the rate of flow.

Reese discloses a method of administering anesthesia directly to the surgical site. The plunger of a spring-loaded syringe creates pressure thus causing the medication to flow through a cannula and catheter into the wound. Flow of the medication is regulated by the micro-bore cannula to ensure delivery at very small rates.

Vaillancourt ('389) teaches an ambulatory infusion pump with a preloaded spring having a fixed spring constant. The preloaded spring is released by a tab and biases the piston of the pump. The biasing force of the spring and the stroke of the piston are coordinated to maintain pressure on the fluid and dispense the fluid at a slow rate.

Ishikawa discloses a medical liquid injector for continuous transfusion. A syringe is fitted with a piston and a cap having an elastic pressing device for continuously pressing the piston to force the liquid from the syringe. Flow is controlled using a flow rate control tube having a given inner diameter.

Elson discloses a fluid delivery system having a bladder enclosed in a cap and drive mechanism. A piston driven by a constant force spring delivers the fluid at a predetermined rate based on the spring design.

Kriesel discloses a fluid container assembly having a plunger that is powered by a stored energy source, such as a compressible cellular mass or an elastomeric membrane, to dispense fluid.

McPhee discloses a syringe actuation device that uses a spring-biased piston.

Ragsdale et al. disclose a spring-powered injection device.

3. Solution to the Problem

None of the prior art references discussed above show a spring-powered infusion pump having a one-way valve (e.g., a duck-bill valve) for retaining the fluid within the dispenser while under pressure from a spring. The spring compressed between the syringe plunger and the syringe cap provides pressure to the plunger so that the fluid contained within the syringe barrel can be released automatically at a continuous rate of flow and the user does not have to manually dispense the fluid contents. A one-way valve retains the fluid within the syringe barrel against the pressure on the plunger from the spring. By inserting an infuser connector through the one-way valve, the fluid retained within the dispenser can be selectively released.

### SUMMARY OF THE INVENTION

The present invention is a spring-powered infusion pump. The spring-powered infusion pump has a syringe barrel with two opposing openings and a plunger disposed between the openings within the syringe barrel. Thus, two chambers are formed between the first opening and the plunger, and the second opening and the plunger. The first opening is fitted with a one-way valve to selectively release fluid retained within the first chamber. The second opening is capped, and a spring is biased between the plunger and syringe cap within the second chamber. The spring applies a force to the plunger in the direction of the first opening. However, the one-way valve retains the fluid within the first chamber, despite the force applied to the plunger, until a tubing set equipped with an infuser connector is attached to the first opening of the syringe barrel. The infuser connector is insertable through the one-way valve and thus provides a passageway for the fluid retained within the first chamber of the syringe barrel. Once attached, the force exerted by the spring causes the plunger to move toward the first opening, thereby dispensing the fluid from the first chamber through the infuser connector and one-way valve into the tubing set.

A primary object of the present invention is to provide a single predetermined dose of fluid at a continuous rate of flow. Inconsistencies in the flow rate associated with manual operation of the syringe plunger are largely eliminated by the spring-powered syringe of the present invention.

Another object of the present invention is to provide a sterile, disposable device for dispensing predetermined amounts of fluid. The syringe barrel of the present invention can be prefilled in a sterile environment so that the fluid is not contaminated prior to being dispensed.

Yet another object of the present invention is to retain the fluid within the syringe barrel prior to being dispensed without the need for caps, clips or other stoppers. When caps or other stoppers are removed, the fluid immediately is released from the syringe barrel, and if tubing is not immediately attached, this fluid is lost and the amount delivered is thus inaccurate. In addition, caps and clips can easily be lost. Fluid is retained within the syringe barrel of the present invention by the one-way valve and released only when an infuser connector fitted within the tubing, is connected to the dispenser. Therefore, when the one-way valve is opened, fluid flows immediately into the tubing and none is lost.

Another object of the present invention is to provide an adjustment mechanism to improve delivery accuracy. A shim or other height adjustment mechanism is used to adjust the spring when manufacturing the infusion pump. The natural variation from spring to spring is reduced or eliminated, resulting in a more uniform driving force in the infusion pump from unit to unit, leading to improved accuracy in the rate of fluid delivery.

These and other advantages, features, and objects of the present invention will be more readily understood in view of the following detailed description and the drawings.

### BRIEF DESCRIPTION OF THE DRAWINGS

The present invention can be more readily understood in conjunction with the accompanying drawings, in which:

FIG. 1 is an exploded perspective view of the spring-powered infusion pump.

FIG. 2 is a cross-sectional view of the assembled infusion pump.

FIG. 3 is a detail cross-sectional view showing an infuser connector penetrating the one-way valve.

FIG. 4 is a detail cross-sectional view corresponding to FIG. 3 after the infuser connector has been completely attached the infusion pump.

FIGS. 5(a) and 5(b) are cross-sectional views of an embodiment of the infusion pump incorporating a series of shims 81 to adjust the force exerted by the spring 60 on the plunger 40.

FIGS. 6(a) and 6(b) are cross-sectional views of an embodiment of the infusion pump incorporating a helical shim 82 that can be trimmed to a desired thickness to adjust the spring force.

FIGS. 7(a) and 7(b) are cross-sectional views of an embodiment of the infusion pump incorporating coaxial beveled segments 83 to adjust the spring force.

FIGS. 8(a) and 8(b) are cross-sectional views of an embodiment of the infusion pump incorporating a jackscrew mechanism 84 to adjust the spring force.

FIGS. 9(a) and 9(b) are cross-sectional views of an embodiment of the infusion pump incorporating a threaded post 85 to adjust the spring force.

FIG. 10 is bottom perspective view of another embodiment of the plunger 40 having a longer skirt and a plurality of raised circumferential ridges 42.

FIG. 11 is a top perspective view of the plunger 40 corresponding to FIG. 10.

FIG. 12 is an exploded perspective view of an embodiment of the infusion pump in which a cap ring 225 on the interior periphery of the cap 50 interlocks with a barrel flange 220 extending about the exterior periphery of the syringe barrel 20 to attach the cap 50 to the syringe barrel 20.

### DETAILED DESCRIPTION OF THE INVENTION

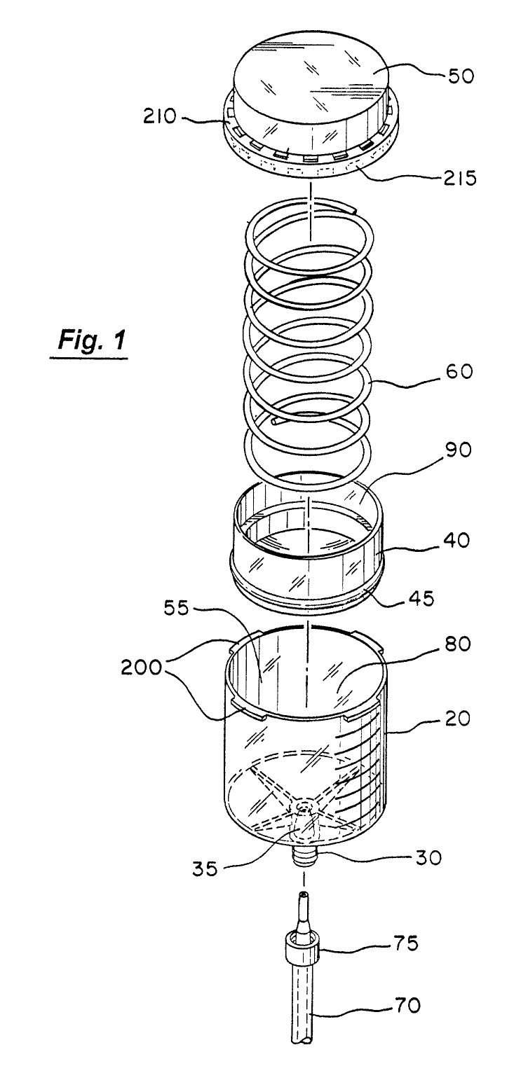

Turning to FIG. 1, the spring-powered infusion pump has a syringe barrel 20 with two opposing openings 30 and 55. The bottom portion of the syringe barrel 20 is tapered to form a dispenser opening 30. Opposite the dispenser opening 30, the syringe barrel 20 forms a top opening 55 that is substantially the same diameter as the syringe barrel 20. A plunger 40 can thus be inserted into, and slidable within, the syringe barrel 20 through the top opening 55. The plunger 40 is surrounded by a circumferential compressible seal 45 (e.g., an o-ring) to form a seal between the plunger 40 and the inside surface of the syringe barrel 20.

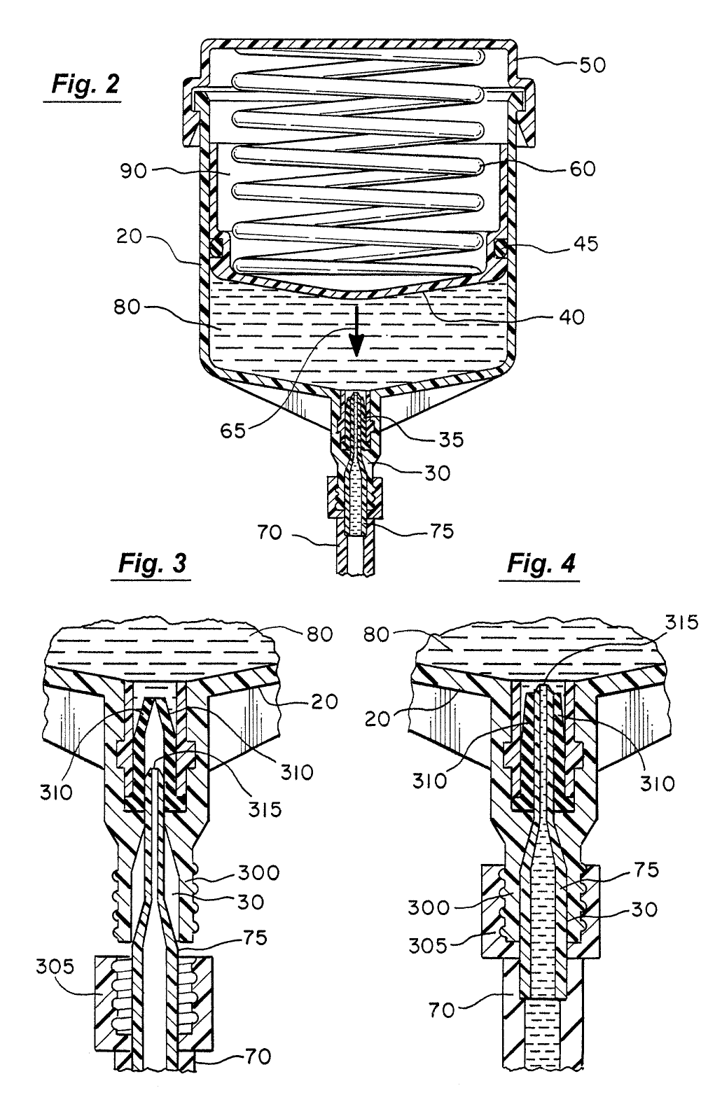

As shown in FIG. 2, a fluid chamber 80 is formed within the lower portion of the syringe barrel 20 between the dispenser opening 30 and the plunger 40. Similarly, as shown in FIG. 1, a spring chamber 90 is formed in the upper portion of the syringe barrel 20 between the top opening 55, which is covered by a cap 50, and the plunger 40. Before the cap 50 is secured over the top opening, a spring 60 is compressed within the spring chamber 90 to exert a force against the plunger 40 in the direction of arrow 65 shown in FIG. 2.

Although in the preferred embodiment the syringe barrel 20 is cylindrical, it is to be expressly understood that the syringe barrel 20, and hence the plunger 40 and compressible seal 45 can be any suitable shape. It is only important that a sealed fluid chamber and a separate spring chamber be formed adjacent one another. In the preferred embodiment, the syringe barrel 20, plunger 40, and cap 50 are made from a hard plastic, such as polypropylene or polycarbonate, so that these parts can be disposed or recycled after use. However, other materials such as metal or glass can also be used for the various components. In addition, the terms “dispenser opening” and “top opening” are intended only to differentiate the two openings, and not to limit the present invention to its orientation.

FIG. 2 shows the present infusion pump after it has been assembled. As depicted in FIG. 1, a series of barrel tabs 200 are formed on the outside of the syringe barrel 20. The cap 50 includes a lip 210 that fits over the top opening 55 of the syringe barrel 20. A series of cap tabs 215 are formed within the lip 210 of the cap 50, which can be snapped over the barrel tabs 200 to securely hold the cap 50 over the top opening 55 of the syringe barrel 20.

In the preferred embodiment, the cap 50 is separate from the barrel 20 to simplify manufacturing and assembly of the present invention. The cap 50 readily fits over the top opening of the barrel 20 and is locked in place. Thus, it is difficult to remove the cap 50 once it is assembled to prevent the cap 50 from popping off due to pressure from the spring 60.

In the preferred embodiment of the present invention, there are more cap tabs 215 than barrel tabs 200 so that the cap 50 and barrel 20 do not have to be perfectly aligned, and so that the cap 50 does not come off under pressure from the spring 60 in the event that cap 50 is rotated. However, it is to be expressly understood that the number and placement of cap tabs 215 and barrel tabs 200 are not important to the present invention so long as the cap 50 can be securely fitted to top opening 55. Likewise, the cap 50 can be secured over the top opening 55 of the syringe barrel 20 in any suitable manner, including but not limited to: permanently bonding the plastic cap to the syringe barrel 20, mechanical latches, or any other suitable design for retaining the cap 50 on the syringe barrel 20 under pressure from the spring 60.

FIGS. 3 and 4 show the details of the one-way valve 35. The preferred embodiment of the one-way valve is a duck-bill, as depicted in FIGS. 3 and 4. However, it is to be expressly understood that any appropriate type of one-way valve may be used, including but not limited to a ball valve, flapper valve, umbrella valve, disc valve, or any other suitable design for allowing flow in one direction while preventing flow in the opposite direction except when pierced or otherwise opened with a mating component. The one-way valve 35 is securely fitted within the dispenser opening 30. The duck-bill embodiment of the one-way valve 35 typically is formed by two “duck-bills” 310 made of a soft, sealable plastic or rubber having an opening between the duck-bills 310. In its normal position shown, the one-way valve 35 is in a closed position (e.g., the duck-bills 310 are collapsed against one another by fluid pressure) so that the fluid is retained within the fluid chamber 80. The dispenser opening 30 includes threads 300 so that the threaded connector 305 of the tubing 70 can be secured to the dispenser opening 30. As the threaded end of tubing 70 is threaded onto the dispenser opening 30, a tubular portion 315 of the infuser connector 75 is inserted between the duck-bills 310, thus spreading duck-bills 310 and forming a conduit from the fluid chamber 80 through the dispenser opening 30 (via the one-way valve 35) and through the infuser connector 75 (via the needle-like portion 315) and into the tubing 70. Thus, fluid retained within fluid chamber 80 is allowed to flow, under the force of the spring 60, into the tubing 70 to be dispensed (e.g., as an IV into a patient).

Note that in the duck-bill embodiment of the one-way valve 35, the duck-bills 310 and infuser connector 75 are designed so that upon insertion of the infuser connector 75, the duck-bills 310 form a seal around the tubular portion 315 of the infuser connector. This seal prevents fluid from leaking through the one-way valve 35 and around the outside of the infuser connector 75.

In the preferred embodiment, the dispenser opening 30 and the tubing connector 305 are threaded. However, any suitable means for securely attaching the tubing 70 to the dispenser opening 30 can be used without departing from the scope of the present invention. For instance, the dispenser opening 30 may be ribbed to receive the tubing 70, or any other suitable design may be used so long as the tubing 70 is held securely to the dispenser opening 30 when the infuser connector 75 opens the one-way valve 35.

In manufacturing springs, there is some degree of variation from spring to spring. In other words, if a group of springs are compressed to the same height, the force exerted by each spring will vary throughout the group. In the embodiment of the infusion pump shown in FIGS. 1-4, the spring will be compressed to the same height (i.e., the distance in the infuser between the cap 50 and the plunger 40). The pressure created in the infusion pump is proportional to the force generated by the spring 60. Since springs vary in force from spring to spring, the pressure created in each infusion pump will vary from unit to unit. Flow rate is proportional to the infuser pressure, so the variation in spring force ultimately leads to variations in flow rate, which is highly undesirable.

To give some indication of the importance of this variation in spring force, infusion pumps are typically required to deliver fluid at a flow rate that is within 15% of an ideal, nominal flow rate. The spring used in current spring-powered infusion pumps varies by about 7%, or almost half of the permissible variation for the entire assembly. If the variation in spring force can be reduced to 2%, for example, we could then have a device with 10% accuracy, instead of 15%. Alternatively, other sources of variation could be more loosely controlled to reduce the cost of the infusion pump.

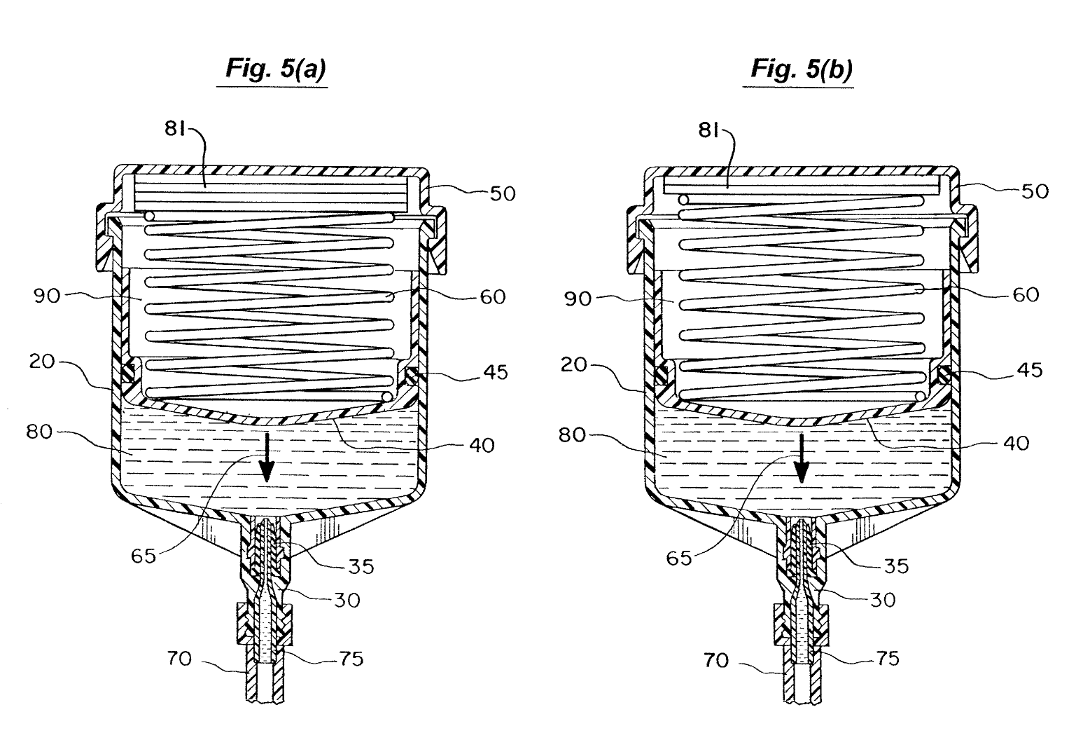

The embodiments shown in FIGS. 5(a) through 9(b) employ shims or other height adjustment mechanisms to adjust the spring compression within each infusion pump to reduce the variation in spring force from unit to unit, thereby effectively reducing variation in flow rate. The further a spring is compressed, the greater the force generated by the spring. If one spring is slightly weaker than another, the same force can be generated by both springs by compressing the weaker spring slightly more.

To illustrate this concept, assume there is a two inch space between the cap 50 and plunger 40. Without any adjustments, each spring will be compressed to two inches once it is assembled into an infuser. Assume the spring rate is 16 lbs per inch. Also assume the average force when compressed to two inches is 40 lbs, but the springs have a 10% variation, so the actual force will vary from 36 to 44 lbs. Using a height adjustment mechanism, all of the springs can be adjusted to 44 lbs by compressing the weaker springs to heights less than two inches. For the weakest springs (i.e., those generating 36 lbs), we need to add another 8 lbs of force. Adding a 0.5 inch shim (so the spring is compressed to 1.5 inches) will increase the spring force to 44 lbs. For an average spring, adding a 0.25 inch shim (so the spring is compressed to 1.75 inches) will add 4 lbs of force to bring those up to 44 lbs, too.

If desired, this approach can be applied to all of the springs in a selected group. However, this approach could be applied to only a portion of the springs, or only those springs falling outside of a predetermined tolerance. For example, adjusting half of the springs would cut variation in half. Adjusting that half of the springs having the worst variations would cut variation by more than half.

FIGS. 5(a) and 5(b) are cross-sectional views of an embodiment of the infusion pump incorporating a stacked series of shims 81 of equal or varying thickness to adjust the force exerted by the spring 60 on the plunger 40. For example, the shims 81 could be shaped as disks or washers.

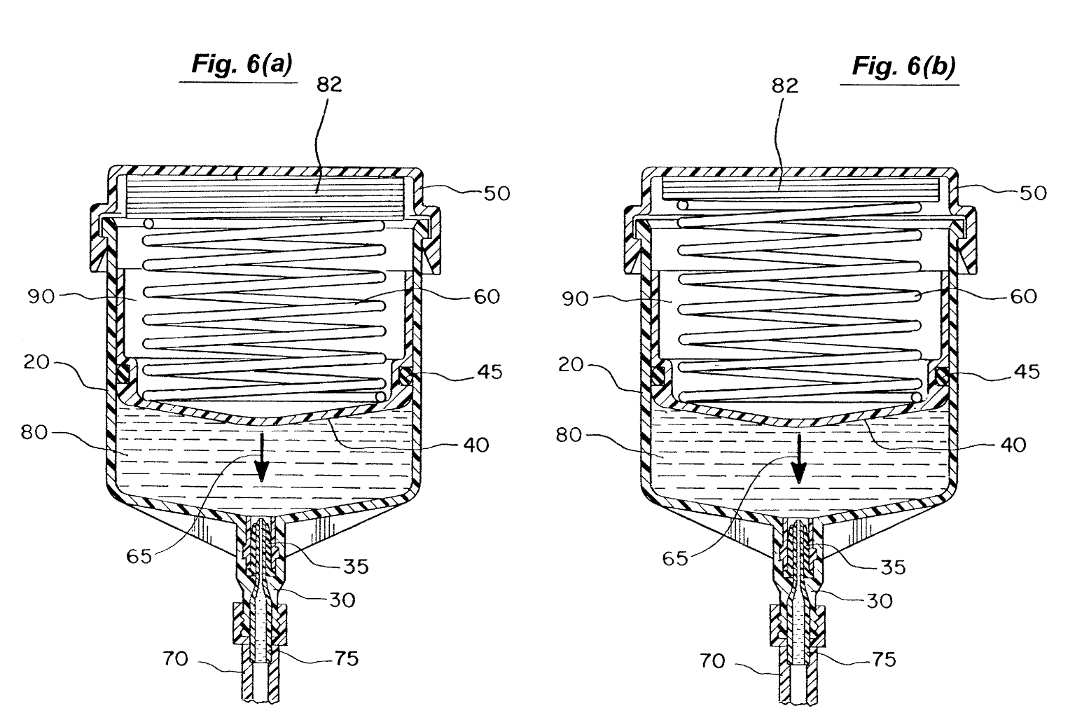

FIGS. 6(a) and 6(b) are cross-sectional views of an embodiment of the infusion pump using a helical shim 82 that can be trimmed to a desired height to adjust the spring force. The manufacturer would simply cut off the proper number of coils to achieve the desired height for the helical shim 82.

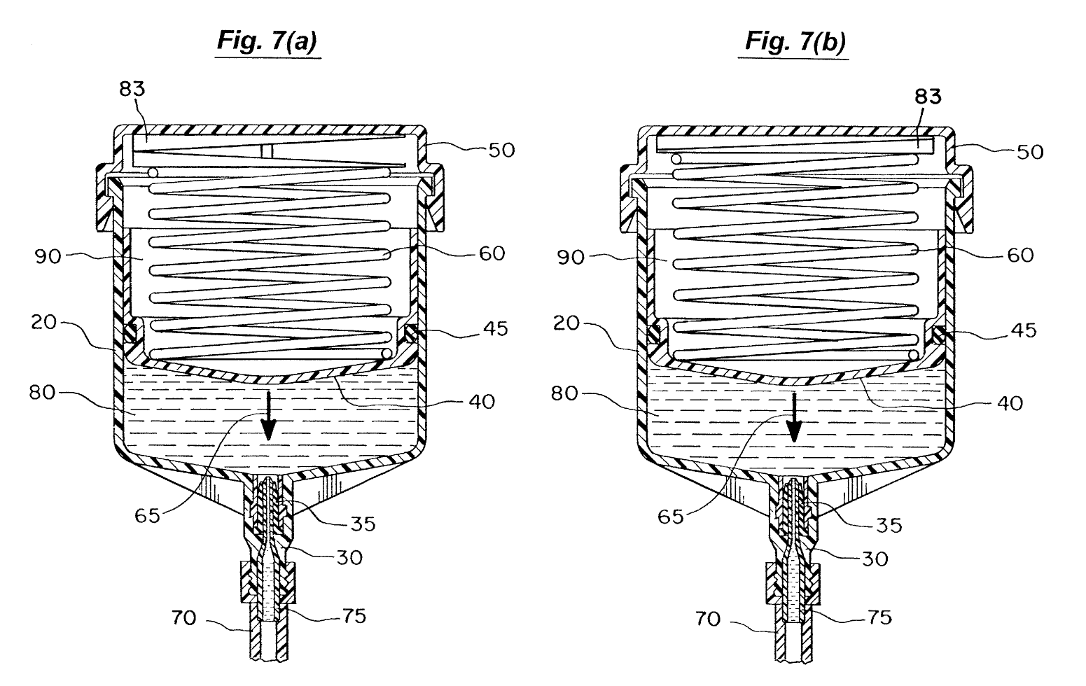

FIGS. 7(a) and 7(b) are cross-sectional views of an embodiment of the infusion pump incorporating coaxial beveled segments 83 to adjust the spring force. This embodiment is similar to the device used to damp a swinging door. Two beveled cylindrical segments are stacked atop one another. Rotating the cylindrical segments with respect to one another increases or decreases the overall height of the assembly.

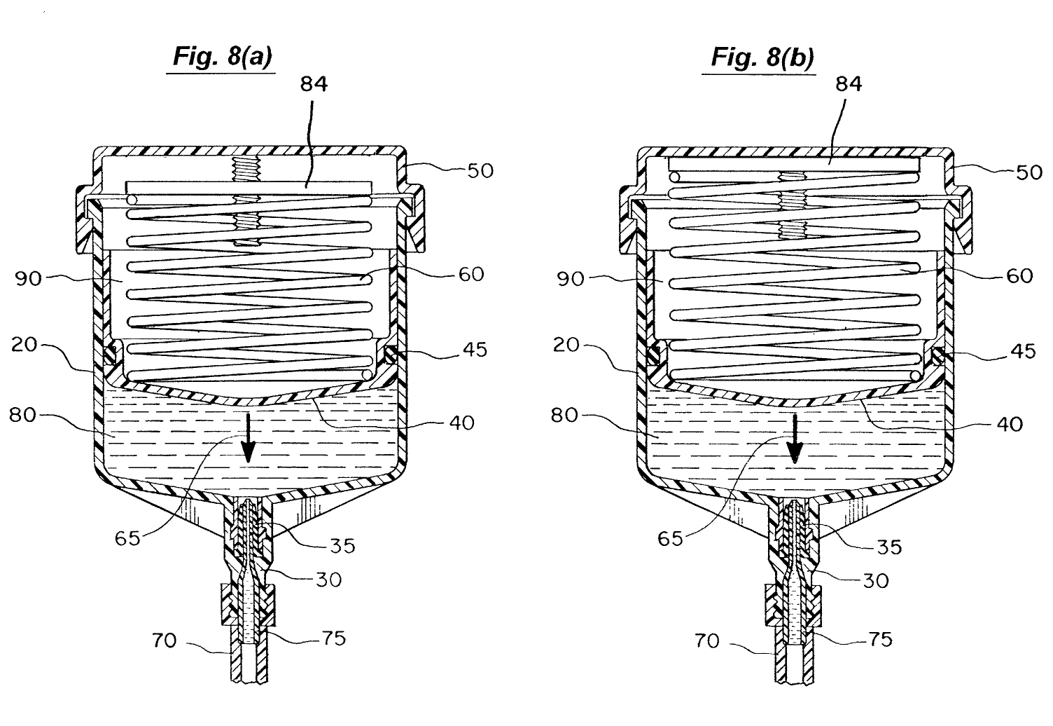

FIGS. 8(a) and 8(b) are cross-sectional views of an embodiment of the infusion pump incorporating a jackscrew mechanism 84 to adjust the spring force. The upper end of the spring 60 abuts a plate threaded on a screw attached to the cap 50 of the infusion pump. Turning the screw or the plate moves the plate up or down to adjust spring compression.

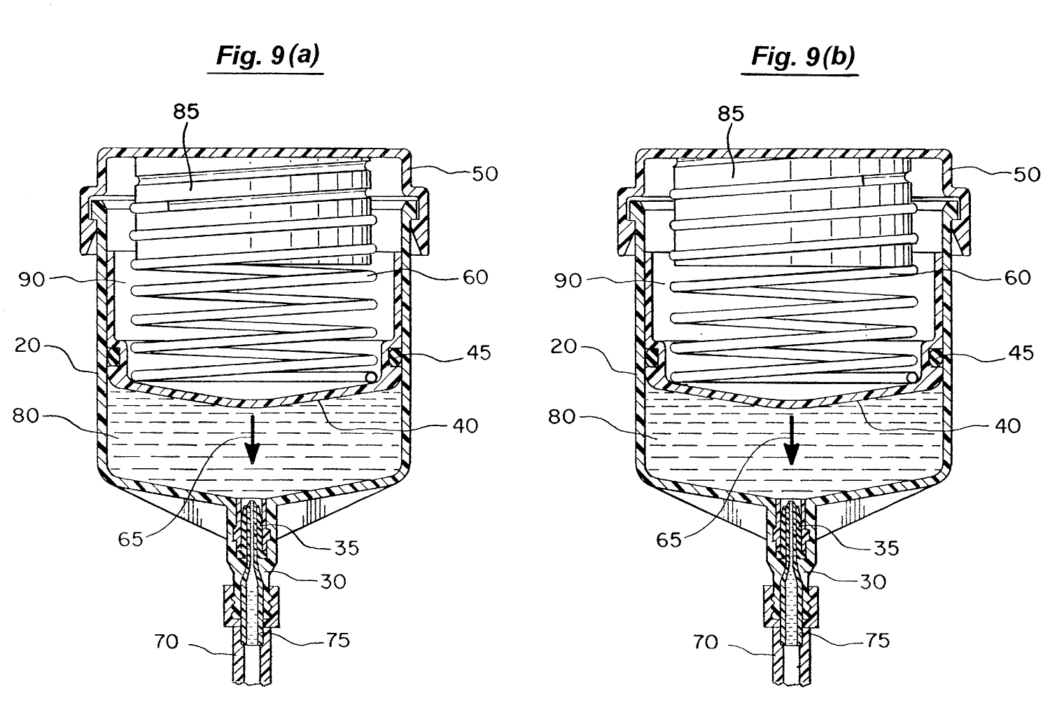

FIGS. 9(a) and 9(b) are cross-sectional views of an embodiment of the infusion pump incorporating a threaded post 85 to adjust the spring force. The spring 60 threads down over the post 85. The effective length of the spring 60 is adjusted by how far it is screwed onto the post 85.

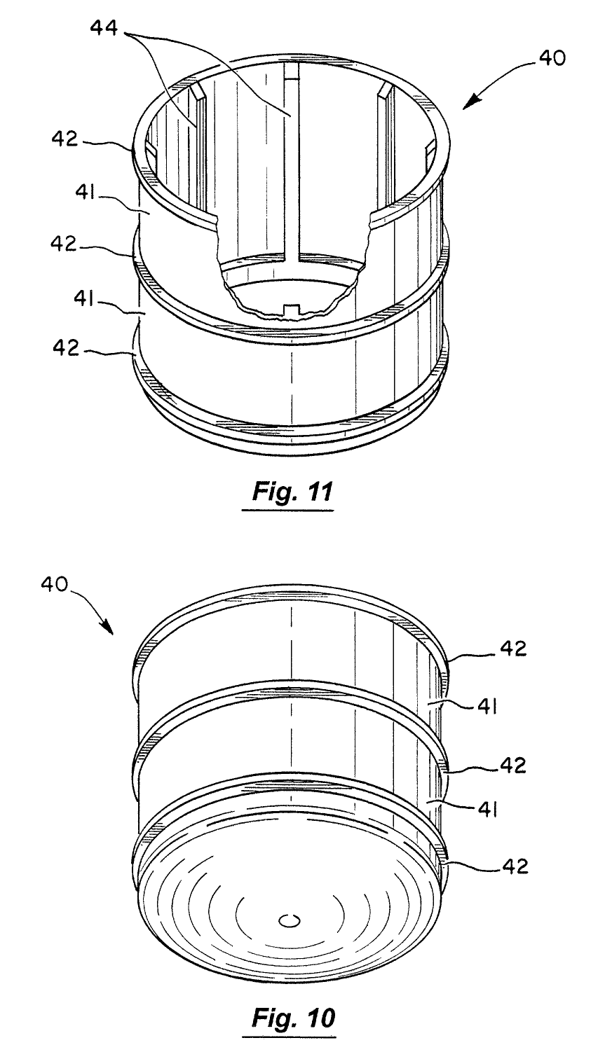

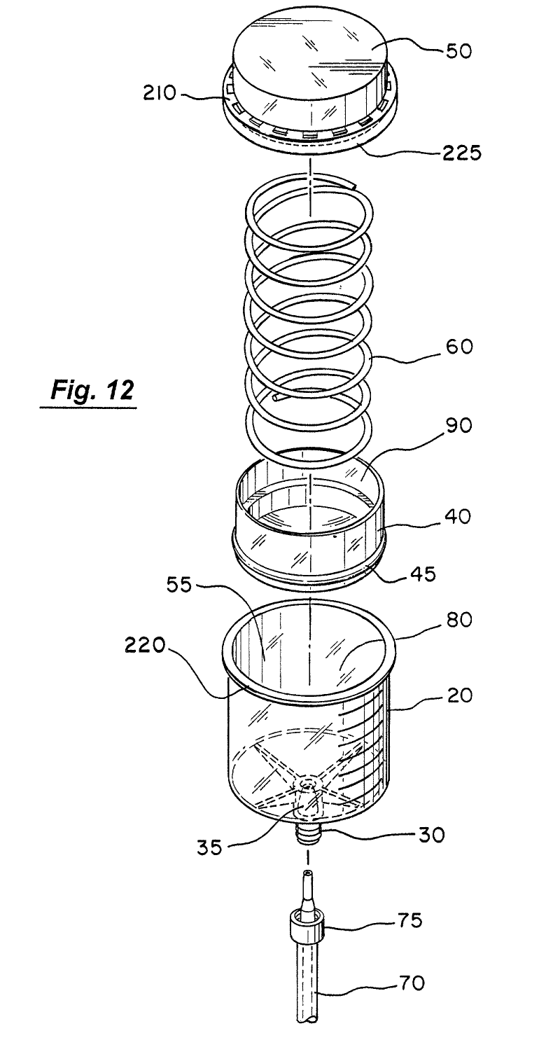

FIGS. 10 and 11 are bottom and top perspective views, respectively, of an embodiment of the plunger 40 having an elongated side wall and a plurality of raised circumferential ridges 42 extending about the periphery the plunger 40. These ridges 42 are separated by recessed areas 41. FIG. 12 is a cross-sectional view of an infusion pump using the plunger 40 from FIGS. 10 and 11. This embodiment of the plunger 40 is less likely to become jammed in the syringe barrel 20, reduces potential friction, and provides a more continuous flow rate.

The embodiment of the plunger 40 shown in FIG. 11 includes a plurality of contoured ribs 44 on the interior surface of the plunger 40. The coils of the spring 60 tend to catch on any exposed edges of the plunger 40, including the upper edge of the plunger 40. This results in uneven friction as each spring coil catches and then slips free. The end results are dips and spikes in the flow rate from the infuser, with flow slowing as a coil catches, and then a spike in the flow rate when the coil slips free. The interior ribs 44 bear on the cylindrical surface of the spring 60. The upper ends of the ribs taper away from the spring 60, thereby eliminating any corners or edges that the spring 60 could rub on.

FIG. 13 is an exploded perspective view of an embodiment of the infusion pump in which a cap ring 225 on the interior periphery of the cap 50 interlocks with a barrel flange 220 extending about the exterior periphery of the syringe barrel 20 to attach the cap 50 to the syringe barrel 20. This configuration helps to ensure that the cap 50 is permanently secured to the syringe barrel 20 and minimizes the risk that the infuser might be accidentally disassembled or tampered with.

Design considerations will determine the rate at which fluid is dispensed from the syringe barrel 20. For instance, the diameter of the dispenser opening 30, the size of the opening created by the one-way valve 35 and the infuser connector 75, the diameter of the tubing 70, and the spring constant of the spring 60 can be selected to achieve the desired flow characteristics. Flow restrictor elements attached to the tubing 70 can also serve to regulate the flow. Likewise, the amount of pressure exerted by the spring 60 will determine the characteristics of the one-way valve 35 required to retain the fluid within the fluid chamber 80. Markings on the syringe barrel can be used to indicate the amount of fluid retained in the fluid chamber 80.

The fluid chamber 80 of the present invention is preferably filled by the manufacturer or the health care provider. The device is assembled with the plunger 40 disposed within the syringe barrel 20 and the spring 60 compressed between the plunger 40 and the cap 50. The health care provider then connects a filling apparatus to the device. Pressurized fluid is fed into the fluid chamber 80 with sufficient force to overcome the force of the spring 60. The one-way valve 35 opens with the applied force of the fluid and allows the fluid to flow into the fluid chamber 80. Once the fluid chamber 80 is filled to a predetermined level, the filling apparatus is removed and the one-way valve 35 returns to its sealed position to retain the fluid within the fluid chamber 80. The fluid is retained by the one-way valve 35 until the spring-powered infusion pump is ready for use, at which time, tubing 70 having an infuser connector 75 is connected to the dispenser opening 30, and the fluid is allowed to flow from the fluid chamber 80 as described above.

The flow rate delivered by the infusion pump gradually slows as it empties due to relaxation of the spring. This is clinically desirable in some applications, such as pain management, in which the patient's need for medication tapers off with time. There may be other applications in which changing the flow rate over time would be desirable. By changing the spring geometry or using multiple springs, compound delivery profiles can be obtained, with step-changes in flow rate or differing flow rates for different portions of the delivery.

It is to be expressly understood that the spring-powered infusion pump of the present invention can be filled by the manufacturer and disposed of after use, or the present invention may be refilled for repeated use. Alternatively, the infusion pump can be shipped empty and filled by the healthcare provider before use. The preferred embodiment offers the advantage of accurate filling and sterility.

Alternatively, the user may wish to fill the infuser with fluid and then freeze it. This is often not feasible with other types of infusers because the fluid expands when frozen and damages the device (e.g., cracks the housing, breaks seals, stretches seals open). The present infuser has additional capacity allowing overfill. If the infuser has not been purposely overfilled, it can be frozen and the plunger 40 and spring 60 will simply move further back to accommodate the increased volume due to expansion upon freezing.

The above disclosure sets forth a number of embodiments of the present invention. Other arrangements or embodiments, not precisely set forth, could be practiced under the teachings of the present invention and as set forth in the following claims.

We claim:

1. A spring-powered infusion pump comprising:
a syringe barrel having a dispenser opening on one end for dispensing liquid contained in the syringe barrel and a top opening opposite the dispenser opening;
a plunger slidably disposed within the syringe barrel forming a fluid chamber within the syringe barrel between the plunger and dispenser opening for receiving a liquid through the one-way valve;
a one-way valve at the dispenser opening to retain liquid within the fluid chamber when the one-way valve is closed, and allowing liquid to flow from the fluid chamber when an infuser connector is inserted through the one-way valve;
a cap attached to the top opening of the syringe barrel, forming a spring chamber adjacent to the fluid chamber within the syringe barrel between the cap and plunger; and
a spring compressed between the plunger and cap within the spring chamber, said spring applying a force to the plunger in the direction of the dispenser opening.

2. The spring-powered infusion pump of claim 1 further comprising:
a tubing set; and
an infuser connector attached to the tubing set, said infuser connector insertable through the one-way valve to form a conduit for liquid flow from the fluid chamber through the tubing set.

3. The spring-powered infusion pump of claim 2 wherein the liquid is released at a substantially continuous flow rate through the tubing set.

4. The spring-powered infusion pump of claim 1 wherein the dispenser opening is threaded to removably secure an infuser connector to the dispenser opening.

5. The spring-powered infusion pump of claim 1 further comprising a height adjustment mechanism adjusting the effective height of the spring within the spring chamber to produce a desired force on the plunger.

6. The spring-powered infusion pump of claim 1 wherein the plunger further comprises a plurality of raised rings extending about the periphery the plunger separated by recessed areas.

7. The spring-powered infusion pump of claim 1 further comprising:
a cap tab on the cap; and
a barrel tab on the syringe barrel, said cap tab and barrel tab interlocking to attach the cap to the top opening of the syringe barrel.

8. The spring-powered infusion pump of claim 1 further comprising:
a cap flange on the interior periphery of the cap; and
a barrel flange extending about the exterior periphery of the syringe barrel, said cap flange and barrel flange interlocking to attach the cap to the top opening of the syringe barrel.

9. A spring-powered infusion pump comprising:
a syringe barrel having a dispenser opening on one end and a top opening opposite the dispenser opening;
a one-way valve at the dispenser opening to retain liquid within the fluid chamber when the one-way valve is closed, and allowing liquid to flow from the fluid chamber when an infuser connector is inserted through the one-way valve;
a plunger movable along the syringe barrel forming a fluid chamber in the syringe barrel between the plunger and dispenser opening, said fluid chamber retaining a liquid therein;
a cap secured to the top opening of the syringe barrel forming a spring chamber within the syringe barrel between the cap and plunger;
a spring compressed between the plunger and cap within the spring chamber, said spring applying a force to the plunger in the direction of the dispenser opening; and
a height adjustment mechanism adjusting the effective height of the spring within the spring chamber to produce a desired force on the plunger.

10. The spring-powered infusion pump of claim 9 further comprising:
a tubing set; and
an infuser connector attached to the tubing set, said infuser connector insertable through the one-way valve to form a conduit for liquid flow from the fluid chamber through the tubing set.

11. The spring-powered infusion pump of claim 10 wherein the liquid is released at a substantially continuous flow rate through the tubing set.

12. The spring-powered infusion pump of claim 9 wherein the dispenser opening is threaded to removably secure an infuser connector to the dispenser opening.

13. The spring-powered infusion pump of claim 9 further comprising:
a cap tab on the cap; and
a barrel tab on the syringe barrel, said cap tab and barrel tab interlocking to attach the cap to the top opening of the syringe barrel.

14. The spring-powered infusion pump of claim 9 further comprising:
a cap ring on the interior periphery of the cap; and
a barrel flange extending about the exterior periphery of the syringe barrel, said cap ring and barrel flange interlocking to attach the cap to the top opening of the syringe barrel.

15. The spring-powered infusion pump of claim 9 wherein the plunger further comprises a plurality of raised rings extending about the periphery the plunger separated by recessed areas.
